(19) United States
(12) Patent Application Publication
(10) Pub. No.: US 2012/0237557 A1
(43) Pub. Date: Sep. 20, 2012

(54) BIOACTIVE CARBON-NANOTUBE AGAROSE COMPOSITES FOR NEURAL ENGINEERING

(75) Inventors: Dan Lewitus, Tel-Aviv (IL); Joachim B. Kohn, Piscataway, NJ (US); Alexander Neimark, Princeton, NJ (US); John Landers, Riverton, NJ (US)

(73) Assignee: RUTGERS, THE STATE UNIVERSITY OF NEW JERSEY, New Brunswick, NJ (US)

(21) Appl. No.: 13/308,510

(22) Filed: Nov. 30, 2011

Related U.S. Application Data
(60) Provisional application No. 61/417,913, filed on Nov. 30, 2010.

(51) Int. Cl. A61K 47/36; A61K 9/00; A61K 38/38; A61K 35/12; B29C 70/06; A61K 38/14; B82Y 5/00

(52) U.S. Cl. 424/400; 264/108; 514/777; 514/769; 514/772.7; 514/772.3; 514/20.9; 514/15.2; 424/93.7; 977/750; 977/752; 977/753; 977/906

(57) ABSTRACT

Nanocomposite fibers containing one or more carbon nanotubes encapsulated in an polysaccharide gel matrix.

### CROSS REFERENCE TO RELATED APPLICATIONS

The instant application claims 35 U.S.C. §119(e) priority to U.S. Provisional Patent Application Ser. No. 61/417,913 filed Nov. 30, 2010, the disclosure of which is incorporated by reference herein.

### STATEMENT REGARDING FEDERALLY FUNDED RESEARCH

This invention was made with government support under Grant R01 EB007467 awarded by the National Institutes of Health. Accordingly, the U.S. Government has certain rights in this invention.

### FIELD OF THE INVENTION

The present invention relates to carbon nanotube agarose based composite materials suitable for tissue engineering applications.

### BACKGROUND

It is generally recognized that cortical neural prosthetic devices are limited to 12 months or less before their recording performance deteriorates substantially. This limitation lies with the fact that a sustained reactive response develops upon insertion of the probe. This response, known as gliosis, diminishes the long-term performance of devices. Control of the brain cell response to the inserted device could lead to improvement of its long-term performance. A number of approaches have been considered, both in terms of biochemistry and design. Examples include the addition of anti-inflammatory agents or cell cycle-inhibiting drugs, as well as surface modification of silicon substrates. Nevertheless, these approaches are burdened by the large stiff constructs that will be present in the tissue throughout its lifetime. To circumvent this, an approach has recently emerged relying on two principals. First, these devices should be made of flexible materials. This will reduce the mechanical disparity between the device and the brain and possibly reduce development of the chronic glial response. Second, devices smaller in size, comparable to the neuronal soma, could lead to a reduction in the chronic glial response through the restoration of neuronal and astroglial synapses. Therefore, smaller and more flexible devices may reduce reactive responses and improve long-term performance, e.g., recording of neural signals.

Carbon nanotubes (CNT) display unique characteristics of superior conductivity, tremendous stiffness and a high aspect ratio. As such, they have been extensively employed in novel materials stemming from their ability to absorb strain and induce conductivity. In addition, it has been shown that macroscopic materials made out of CNT are in fact biocompatible, making their inclusion into materials destined for medical applications that much more desirable. Additionally, the incorporation of carbon nanotubes maintains a material's structural stability during cell growth. This attribute is coupled with the fact that CNT can support neuron cell growth and differentiation, a decisive factor for any device that hopes to induce electrical stimulation with neurons in vivo.

This evolving interest in natural polymers destined for drug delivery and tissue engineering has led to the emergence of new hybrid materials. So far a popular method to fabricate CNT/polymer hybrids is through the technique of wet spinning. Wet spinning has been utilized in producing CNT/polymer composite fibers for the last 10 years. Despite its inherent advantage, the ability to scale up the production of CNT fibers using the wet spinning technique incurs some drawbacks. These drawbacks are observed where a polymer, such as PVA, is utilized as the bath component versus when it is used as the dispersant. The former leads to several shortcomings that make the process difficult to scale commercially. The primary concern arises when the gel ribbon becomes suspended at the spinning position. To prevent the ribbon from clashing into itself, it is necessary to continually raise the tip of the spinning bath.

With the removal of the polymer from the bath, however, there is a reduction in several degrees of freedom inherent to how the polymer solution is prepared and time of coagulation. This in turn makes the process less complex. Several authors have demonstrated this practicality by using the polymer as the dispersant. See e.g., A. J. Granero et al., Adv. Funct. Mater. 2008, 18, 3759. This provides several advantages, including the fact that the spun ribbon can be reeled up onto a spool and the polymer can be used much more effectively. Alternative methods have been proposed that lead to a cleaner product and less expensive process, including the use of polymeric hydrogels. The advantage of such hydrogels is owed in part to their ability to imitate the natural extra cellular matrix (ECM), thus promoting cell growth. Another advantage of using the polymer as a dispersant is that deciphering the composition of the fiber becomes easier as it is only dependent on the initial concentrations of the dispersion. This is contrary to analyzing the fiber post facto when it is spun into a polymer bath. When using that method, the composition of the fiber will be dependent on the polymer concentration and adsorption kinetics.

When using the polymer as a dispersant, CNT are dispersed with the aid of a surfactant or polymer by non-covalent means. Some of the current polymers that aid in the production of CNT, especially those specifically designed to be biologically viable, are based on the use of natural polymers or naturally based dispersant that are known to be biocompatible, such as chitosan, hyaluronic acid, DNA and chondroitin sulfate.

### SUMMARY OF THE INVENTION

One aspect of the present invention is directed to a biocompatible nanocomposite fiber containing one or more carbon nanotubes encapsulated in a gel matrix of a polysaccharide such as agarose. In certain embodiments, this biocompatible carbon nanotube-based fiber may have a functionalized surface that allows for covalent attachment of one or more bioactive substances. Bioactive substances may be selected from proteins, peptides, glycogens and drugs. Examples of these bioactive substances include laminin, alpha melanocyte stimulating hormone, and L1 cell adhesion molecule. Additionally, in certain embodiments, the fiber is loaded with at least one particle selected from the group consisting of platinum, palladium, gold, silver, titanium nitride, tantalum, tantalum oxide, iridium oxide and conductive polymers such as poly(3,4-ethylene-dioxythiophene), polyimide, polyanyline, and polypyrole.

In another aspect, the present invention is directed to a method for fabricating a biocompatible carbon nanotube-based fiber, by: (1) preparing a liquid dispersion solution comprising carbon nanotubes and a polysaccharide such as agarose; (2) injecting the liquid dispersion solution into a rotating bath of ethanol; and (3) drying the pre-fibers. In another embodiment, the fibers may be fabricated by: (1) preparing a liquid dispersion solution comprising carbon nanotubes and a polysaccharide such as agarose; (2) injecting the liquid dispersion solution into a tube; (3) allowing the liquid dispersion to form a molded gel in the tube; and (4) removing the molded gel from the tube.

In yet another aspect, the present invention is directed to a method for delivering a desired biomolecule to a subject comprising the steps of loading the biocompatible carbon nanotube-based fiber of the present invention with a desired biomolecule; and contacting a subject to which the biomolecule is to be delivered with the carbon nanotube-based fiber. In certain embodiment, the loading of the polymeric nanoparticle carrier comprises covalently attaching the desired biomolecule to the agarose. In certain embodiments, the desired bio-molecule is a drug.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows Scanning Electron Microscopy (SEM) images of CNT agarose fibers. The images on the left display a cross section of a molded fiber (FIG. 1a), a close up of the molded fiber body depicting the smooth morphology of the surface (FIG. 1b), and a close up of the cross section of the molded fiber depicting the carbon nanotube bundles (FIG. 1c). The images on the right display a cross section of the wet spun agarose fiber (FIG. 1d), a close up of the wet spun agarose fiber body depicting the rough morphology (FIG. 1e), and a close up of the cross section wet spun agarose fiber depicting the carbon nanotube bundles (FIG. 1f).

FIG. 2 shows Transmission Electron Microscopy (TEM) images of molded fibers demonstrating fiber orientation in the direction of molding indicated by the arrows.

FIG. 3 displays a merged fluorescent and phase contrast image of BSAC− conjugate control fiber (FIG. 3A), a merged fluorescent and phase contrast image of BSAC+ conjugate functionalized fiber (FIG. 3B) and a fluorescent image LN+ laminin functionalized fiber (FIG. 3C). The exposure time to the fluorescent channels were kept constant to eliminate gain variability and false images. Fluorescent intensity (FI) readings were taken from fibers placed in a well plate then scanned through a plate reader, the results of which are shown in FIGS. 3D and 3E.

FIG. 4A displays cell viability after exposure to four types of fibers. The data is plotted against positive control. FIG. 4B shows projected phase contrast and fluorescent images of DAPI stained fixed astrocytes grown on LN+ disc. The edge of the disc is marked by white arrows. Cells are solidly attached to only the agar disc. FIG. 4C shows a projected confocal image of live astrocytes grown on LN+ stained with Calcien AM.

FIG. 5 shows representative immunohistochemical images of fibers inserted into rat cortex. Yellow—astrocytes (GFAP). Blue—microglia (Iba-1). Green—neurons (Nissl). Scale bar 200 μm. FIG. 5A displays an image of a CDAP+ fiber. FIG. 5B displays an image of an LN+ fiber. FIGS. 5C, 5D, and 5E provide normalized intensity of cell expression at the fiber vicinity for microglia, astrocyte, and neuron respectively.

FIG. 6 shows projection confocal images of fibers extracted from brains. Images are of two sides of each fiber mounted on the glass slide (designated as LN− and LN+). Yellow—astrocytes (GFAP). Blue—microglia (Iba-1). Green—Neurons (Nissl). The micrograph of the laminin functionalized fiber (LN+, FIGS. 6C and 6D) demonstrates a greater attachment of all cell types when compared to non-functionalized fiber (LN−, FIGS. 6A and 6B). Non-specific cell attachment is more evident with the LN+ fibers.

FIG. 7 shows fluorescent microscopy images of SulforhodamineB (hydrophilic) fibers before (left) and after (right) release.

FIG. 8 shows fluorescent microscopy images of 5-Dodecanoylaminofluorescein (hydrophobic) loaded fibers, before (left) and after (right) release.

FIG. 9 shows projected confocal images depicting glial (Iba—microglia, GFAP—astrocytes), neural (NeuN) response and cell attachment to pristine and α-MSH functionalized CNF electrodes.

FIG. 10 shows ultrathin SWNT/agarose fibers produced by wet spinning that are approximately 26 μm in width.

### DETAILED DESCRIPTION OF PREFERRED EMBODIMENTS

The present invention relates to a novel approach for producing nanofiber composites of carbon-nanotube fibers (CNF) in a matrix of a polysaccharide such as agarose. Current attempts to make CNF require the use of a polymer or precipitating agent in the coagulating bath that may have negative effects in biomedical applications. One aspect of the present invention provides that by taking advantage of the gelation properties of polysaccharides such as agarose, one can substitute the bath with distilled water or ethanol and hence reduce the complexity associated with alternating the bath components or the use of organic solvents. Another aspect of the present invention provides that these CNF can be chemically functionalized to express biological moieties through available free hydroxyl groups in agarose. The resulting CNF are not only conductive and nontoxic, but their functionalization facilitate cell attachment and response both in vitro and in vivo. A further aspect of the present invention is the use of the CNF for localized drug delivery. The agarose/carbon nanotube (CNT) hybrid materials of the present invention are thus excellent candidates for applications involving neural tissue engineering and biointerfacing with nervous system, including, but not limited to, use as regenerative nerve conduits, intrafascicular electrode, and cortical neural probes.

The present invention combines three elements that have not yet been adjoined: (1) the ease of wet spinning as a fabrication technique, (2) the reinforcing and conductance properties of CNT and (3) the gelation and functionalization potential of polysaccharides such as agarose. This combination creates a continuous electro and neuron conductive biohybrid nanocomposite fiber.

Those of ordinary skill in the art guided by the objectives of the present specification will recognize CNT suitable for use with the present invention. The present invention does not require a specific CNT. However single-wall carbon nanotubes (SWNT) are preferred.

Agarose is an algae derived linear polysaccharide hydrogel possessing a sub-micron pore structure. It is a poly(1→4)-3,6-anhydro-α-1-galactopyranosyl-(1→3)-β-d-galactopyranose) with thermoreversive properties. Although it is a non cell adherent, due to its benign and biocompatible nature it is commonly used as a non adhesive substrate for in vitro cell studies.

In addition, agarose has several distinct advantages over other natural polymers. First, its thermal dependant hydrogel properties allow it to be easily malleable into different shapes and forms without the use of additional reagents or organic solvents. Second, unlike extracellular matrices based polymers, specific proteins or DNA, agarose lacks native ligands and is thus inert to mammalian cells. Third, through available primary and secondary hydroxyl groups, agarose can be chemically modified leading to functionalization through grafting of proteins, peptides and glycogens to the polysaccharide backbone, allowing it to be specifically tailored for various biorelevant applications. Fourth, the addition of such molecules can alter not only biocompatible properties, but its mechanical properties as well. Fifth, its high surface to volume ratio and porosity combined with its hydrophilic nature allows for a more effective penetration of cells during seeding while also supporting delivery of nutrients and metabolites to the cells. Carrying out such modifications results in a substantial increase in cell attachment, continuous support of 3D neural cell cultures, the ability to orient cell migration, and specifically enhance neurite extension with the grafting of neuron conductive constituents such as laminin or various oligopeptides. Sixth, unlike other biopolymers, it is non-biodegradable, and, therefore will allow for long term performance and integration of the carbon nanotubes and avoid disintegration of the fabricated structures. And, seventh, agarose is a cheap and abundant polysaccharide, sourced from plants (algae) and can be grown in highly controlled environments.

While agarose is preferred, essentially any polysaccharide with one or more of the foregoing advantages of agarose over other natural polymers may be used. For purposes of the present invention, the term “agarose” is defined as including those polysaccharides. Accordingly, the following description with reference to agarose should not be interpreted as limiting the invention only to the use of agarose as the polysaccharide.

According to different embodiments of the present invention, nanotube fibers were fabricated by two methods, wet spinning and molding the fiber in a hollow tube. Both approaches produce fibers from aqueous dispersions containing CNT and agarose. The dispersions typically contain between about 0.01 and about 20 wt % CNT, more typically between about 0.5 and about 2.5 wt %, and even more typically about 1 wt % CNT. Agarose is used at a level typically between about 0.5 and about 6 wt %, more typically between about 1 and about 5 wt %, and even more typically about 2 wt % agarose.

The amount of agarose should be equal to or exceed the amount of CNT used, typically in a ratio between about 1.1:1 and about 5:1 of agarose to CNT, more typically in a ratio between about 1.5:1 and about 3:1 and even more typically in a 2:1 ratio or agarose to CNT.

The aqueous dispersions are prepared by sonication. During the sonication process, enough heat is generated to invoke the transition of the agarose from an insoluble powder to a viscous liquid. This allows the agarose present in the liquid state to form random coils and physically wrap around and disperse the CNT without the use of additional dispersants such as a surfactant. Any other heating process that produces the same result may be used.

For wet spinning, the liquid dispersion of nanotubes and agarose is injected through a narrow orifice into a rotating bath, with the rotation velocity greater than the velocity at which the dispersion is injected. A solvent in which the agarose dispersion will gel upon cooling is used, such as ethanol. Upon entering the bath, the dispersion displays an axial diffusion which is inhibited by two factors. First, the stretching imposed by the rotating velocity field and second by the gelation of the agarose/CNT composite. By controlling the speed and the rheology of the injecting dispersion and the rotating solution, the width and morphology of the fiber precursor can be controlled. Therefore, a greater rotation speed results in better alignment of the CNT encapsulated in the agarose gel matrix.

For hollow tube molding, CNF are fabricated by injecting the dispersion into a 1 mm diameter tube and allowing it to gel by cooling. The molded gels are then flushed out with lukewarm water.

Wet spinning produces fibers up to 100 m in length having a width between about 10 microns and about 250 microns. Hollow tube molding produces fibers up to 100 m in length having a width between about 10 microns and about 250 microns.

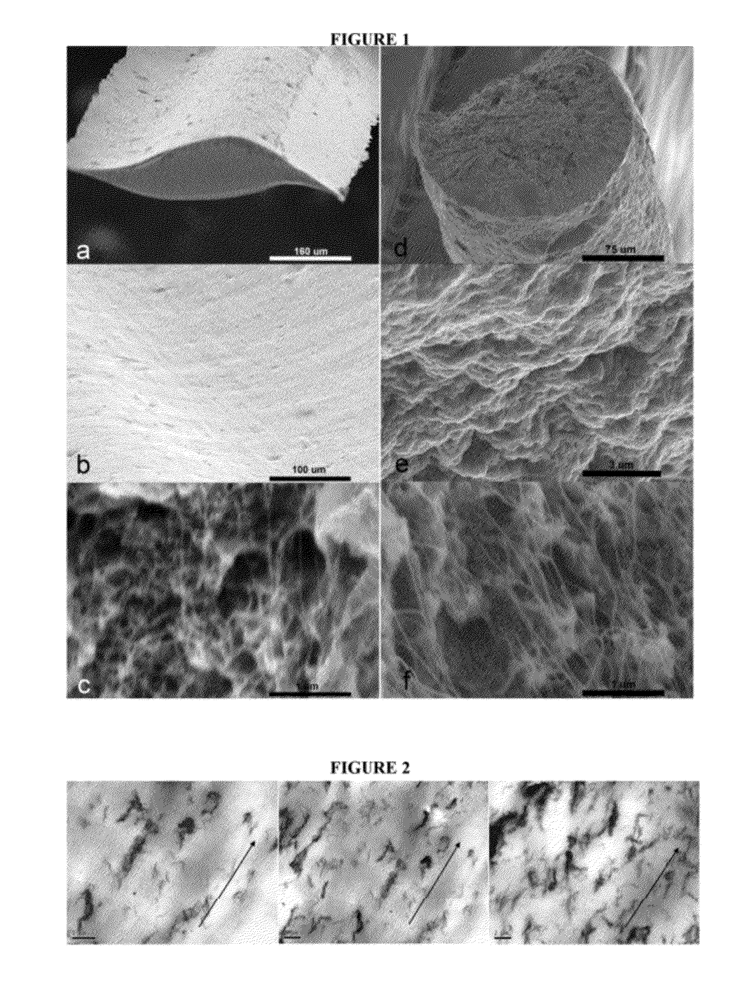

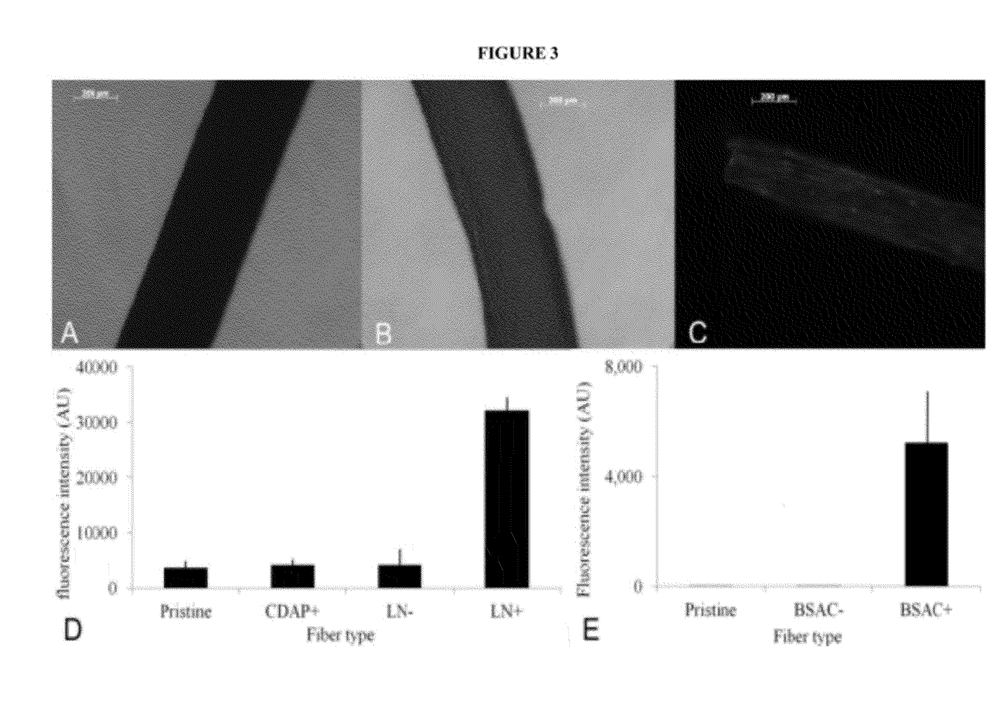

SEM images of molded nanotube fibers are presented in FIGS. 1a, 1b and 1c. This fabrication technique results in a smooth and nearly flat morphology. However, fibers fabricated by the wet spinning method (FIGS. 1d, 1e and 1f) resulted in round circular fibers with a rough outer surface. This is the result of the extraction process from the bath where capillary forces fold the fiber precursor. This ability to control the surface roughness is a key parameter that affects the quality of cellular interfacing between CNF's and cultured neurons. For both types of fibers, a close inspection of the cross section shows the exposure of carbon nanotube bundles depicted in FIGS. 1c and 1f evident by the long overlapping strands. A degree of alignment is still obtained when molding is used, induced when the dispersion is first injected into the tube, as evidenced by the TEM images shown in FIG. 2 in which longitudinal cross sections of CNF fibers demonstrate general orientation in the direction of the fiber.

Another embodiment of the present invention relates to the use of such inherently conductive fibers as microscale neural recording devices in the central nervous system (CNS). They can advance the field of neural prosthetics through long-term biocompatibility and performance allowing the recording devices to interface with brain tissue, for the enhancement of neural integration and the reduction of gliosis formation.

The materials characterized by the present invention function in the peripheral nervous system (PNS) as well. These fibers can be developed into intrafascicular electrodes, thus allowing for neural interfacing with the advantage of being both mechanically compliant and biologically attractive for long-term recording. Additionally, in the PNS, nerve guidance conduits could be prepared either through molding of agarose/CNT dispersions, or as fibers braided into nerve guide conduits where their potential to support nerve growth and regeneration through electrical stimulation, porosity, and biochemical cues is advantageous.

In certain embodiments, the fibers can be loaded with various nanoparticles to either increase the conductivity of the fibers or to increase the capacitance through the use of nanoparticles that exhibit pseudo-capacitance behavior through fast and reversible Faradaic (redox) reactions at the surface. In the former, this includes noble metal nanoparticles such as platinum (Pt), palladium (Pd), gold (Au), silver (Ag), and non noble metal nanoparticles such as titanium nitride (TiN), tantalum and tantalum oxide. The latter includes iridium oxide and conductive polymers such as poly(3,4-ethylenedioxythiophene) (PEDOT), polyimide (PI), polyanyline (PANi), and polypyrole (PPy).

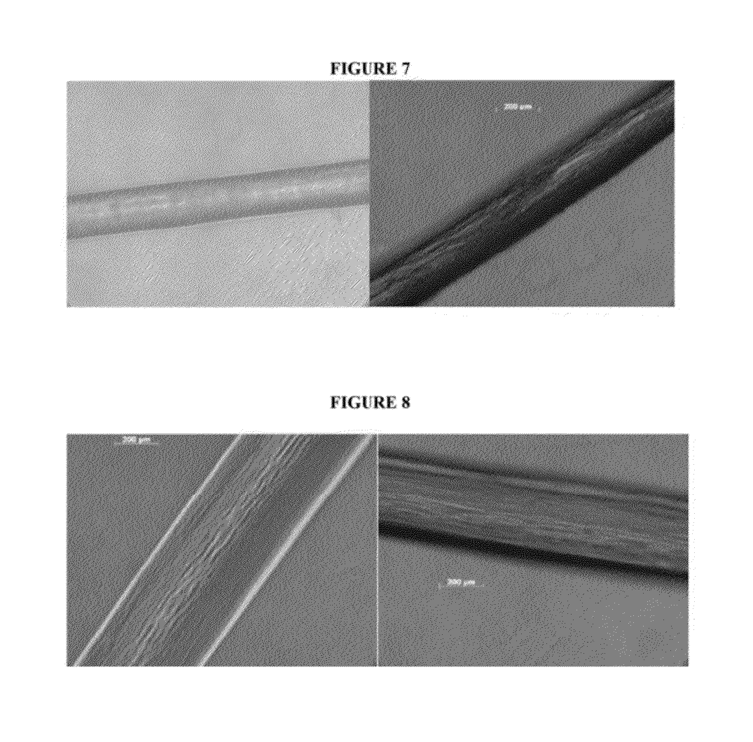

Another embodiment of the present invention relates to use of the carbon-nanotube agarose based composite material for drug delivery. Drugs such as dexametahsone can be absorbed to these materials to allow for localized delivery. To evaluate whether drugs could be locally delivered using the agarose carbon composites, two fluorescent drug models, hydrophobic and hydrophilic, were loaded during the fabrication process to agarose fibers with or without carbon nanotubes. The release of these moieties from the fibers into buffer was visualized using fluorescent microscopy. SulforhodamineB (hydrophilic) fibers before and after release can be seen in FIG. 7. 5-Dodecanoylaminofluorescein (hydrophobic) loaded fibers, before and after release can be seen if FIG. 8.

The following non-limiting examples set forth hereinbelow illustrates certain aspects of the invention.

### EXAMPLES

### Fiber Fabrication

All chemicals were of reagent grade or higher. For both approaches, fibers were produced from a dispersion containing 1 wt. % of SWNTs (Unidym or Nanoledge), 2 wt. % agarose (15517-014, Invitrogen,) and 97 wt. % distilled water. For the first approach, the dispersion was prepared with the aid of a horn sonicator (Mixsonix 5400) for 10 minutes at a pulsed rate of one second on and one second off. The sonicator was operated at 40 amperes. During the sonication process, enough heat is generated to invoke the transition of the agarose from an insoluble powder to a viscous liquid. This allows the agarose present in the liquid state to form random coils and physically wrap around and disperse the SWNT without the use of additional dispersant such as a surfactant. While the dispersion is still a liquid, it is injected through a 1 mm diameter tip into a bath of ethanol at room temperature rotating at a rate of 33 rpm, at which time it becomes a pre-fiber.

The second approach produces 200 μm fibers fabricated by injecting the dispersion into a 1 mm diameter tube and allowing it to gel. The subsequential molds are then flushed out with lukewarm water. Upon drying, these fibers shrink to ribbons 200 μm wide.

Morphology of the fibers was evaluated using a Hitachi S-4500 Field emission SEM. Fresh cut sections were obtained by breaking the fibers after immersion for one 1 minute in liquid nitrogen. This process avoid smearing of the polymer/CNT nanostructures. The orientation of CNT in molded fibers was visualized using transmission electron microscopy. Fibers were embedded in embedding media (Electron Microscopy Sciences) and sectioned longitudinally with a diamond knife (Ultracut E ultramicrotome) at room temperature. Thin sections were applied on a copper Formavar/carbon coated grids (Electron Microscopy Sciences). Electron micrographs were taken using a model JEM 100 CX transmission electron microscope (JEOL).

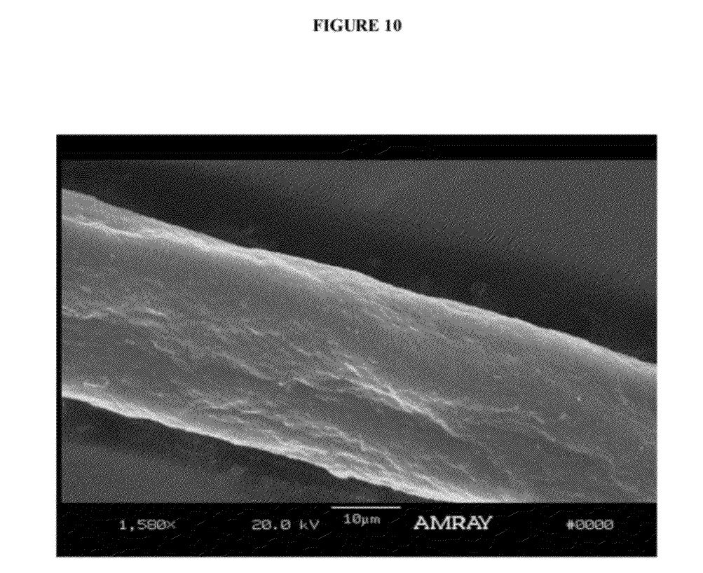

Additionally, ultrathin fibers comprised of SWNT and agarose were produced by wet spinning using a 50 μm tapered microbore (Fisnar). The thinner fibers are comparable to cellular dimensions and thus are advantageous due to their ability to circumvent a foreign body response by the lack of insertion trauma. The fibers are between 15 to 30 μm in diameter. These fibers are displayed in FIG. 10. Such thin fibers may reduce the insertion trauma and, as such, be advantageous compared to more thick fibers.

### Agarose Fiber Activation

CDAP activation of agarose and protein attachment was based on methods published by Kohn and Wilchek (J. Kohn, M. Wilchek, Applied Biochemistry and Biotechnology 1984, 9, 285) with slight modifications: Agarose CNT ribbons were weighed (approximately 4 mg) and placed in a 20 mL glass scintillation vial (Fisher). 10 mL of each of the following solutions were added to the vials; each for 15 minutes followed by aspiration and replacement with the next solution under gentle agitation: (1) Deionized water (twice), (2) 30% acetone (twice), (3) 60% acetone (twice). The last solution was then replaced with 3 mL of ice-cold 60% acetone. Under agitation, 300 μL of 100 mg/mL of CDAP (Sigma) in dry acetonitrile (Sigma) was added. After one minute, 250 μL of 0.2 M Et₃N (Sigma) solution was added drop wise over one minute. After five minutes of mixing, the solution form the vial was aspirated and transferred to a clean vial for activation verification. 5 mL of ice cold 0.05 N HO was added to the fibers for five minutes mixing, followed by five minutes in 5 mL cold deionized water.

Functionalized and control fibers were qualitatively evaluated by both fluorescent microscopy and fluorescent intensity reading. Representative fluorescent and phase contrast images of functionalized (“protein”+) and control fibers (“protein”−) are shown in FIG. 3. Fluorescein conjugated bovine serum albumin (BSAC) allows for direct attachment verification. Because the protein has a fluorescent marker conjugated, its covalent attachment will result in fibers with inherent fluorescence. Therefore, functionalized fibers demonstrate high fluorescence, compared to the control fiber (FIGS. 3A & 3B). The validation of laminin attachment to the agarose carbon nanotube fibers was performed using an immunohistochemical (IHC) technique as shown in FIG. 3C. This method allowed not only validation of the attachment, but also confirmed the retention of the protein conformation, as the primary antibody used is specific for laminin. Moreover, the immunofluorescence of the fibers shows that the agarose orientates itself longitudinally with the fiber. This feature is due to the elongation of the dispersion when it experiences the rotating velocity field during the fabrication process.

Nanotube fibers were placed in a black 96 well plate and tested for fluorescence intensity using a plate reader. Results for LN and BSAC functionalized fibers and their prospective controls are shown in FIG. 3D and FIG. 3E respectively. The control and pristine fibers exhibited low values of fluorescence intensity (FI) with no statistical difference between them (P>0.05). The functionalized fibers FI values were 2 orders of magnitude higher than those of the other two types (P<0.05), indicating successful functionalization. These findings emphasize the advantage of using agarose. It provides a “clean slate” for biochemical manipulation. This allows for specific cellular cues and even several different cues to be covalently conjugated to the fibers, resulting in functionalized material, thus allowing for specific use and application.

### Protein Attachment

Functionalized fibers were added with 5 mL of 20 ug/mL of either laminin (LN) from Engelbreth-Holm-Swarm murine sarcoma basement membrane (L2020, Invitrogen) or fluorescein conjugated bovine serum albumin (BSAC, A23015, Invitrogen) both in 0.1 M NaHCO₃ for at least 16 hours. Remaining active groups were quenched by adding 150 μL of ethanolamine (Sigma) per 100 μL of attachment solution then stirring for 4 hours. Fibers that underwent the full reaction were designated either “LN+” or “BSAC+”. Control fibers designated “LN−” or “BSA−” did not undergo the CDAP addition step but were added with the proteins. Another control group that was not added with any proteins and was designated “CDAP+”, while the pristine fibers were designated as such.

### Washing

Fibers were washed in 10 mL for 15-20 minutes in each of the following solutions: (1) deionized water (twice), (2) 0.5 M NaCl (twice) (3) deionized water (twice). Fibers were then dried in nitrogen, sealed in airtight bags and refrigerated until use.

### Activation Verification

Qualitative verification of the activation of the agarose was performed as described by Kohn and Wilchek Kohn, M. Wilchek, Applied Biochemistry and Biotechnology 1984, 9, 285). 0.15 g of 1,3-dimethylbarbituric acid (Sigma) were dissolved in 9 mL pyridine and 1 mL deionized water. 2 mL of the resulting solution was added with 100 pt of the activation solution.

### Protein Attachment Verification

Visualization of the fibers using a fluorescent microscope was performed. Fibers functionalized with BSAC, control fibers, and pristine fibers (those that did not undergo any reaction) were placed in either a clear or a black 96 well multi-well plate. The clear plate was placed within an inverted fluorescent microscope (Axio Observer-D1, Carl Zeiss MicroImaging GmbH) and imaged using a 10× objective. All fluorescent images were taken with similar exposure time to provide a true reflection of the intensity of the fluorescence. Fluorescent intensity recording from the black plate was taken using a well plate reader (M 200, Tecan). To allow background subtraction from the polypropylene, the fluorescence intensity of empty wells was measured and their average was subtracted from the readings of the fiber containing wells. The mean and standard deviations of fluorescent intensity (FI) measured using constant gains are presented in arbitrary units.

To ensure laminin activation, 5 mm pieces of each type of fiber were placed in a 48 well plate (4 fibers per condition). Wells were added with 300 μL of phosphate buffer saline (PBS, Sigma Aldrich) containing 1% w/v of non-specific blocking serum (BSA, Sigma Aldrich) then gently shaken for 30 minutes. The solution was aspirated followed by 3 washes of the plates with 500 μL of PBS. 300 μL of 1:100 dilution of rabbit polyclonal to laminin primary antibody (ab11575, Abcam) in PBS containing 1% BSA was added to each plate and incubated in room temperature overnight under gentle agitation. Wells were washed three times with 500 μL of PBS, and 300 μL of 1:50 dilution of secondary antibody, Tetra-methylrhodamine goat anti-rabbit IgG (T-2769, Invitrogen), was added to each well and incubated in room temperature for 4 hours under gentle agitation followed by 5 washing steps and a final aspiration. The plate was kept in a dark and dry environment to allow evaporation of excess moisture. Fluorescent images and intensity reading of the fibers were taken as described for the BSAC functionalized fibers.

### Conductivity Measurements

Fibers were partitioned into three batches based on whether CDAP and/or LN were added to the reaction. Within each batch three fibers were tested. Prior to testing, each end of the fiber was dipped in liquid nitrogen and clipped to expose a rigid cross section. Droplets of a gallium-indium eutectic (liquid metal) was placed on each end of the fiber and resistance was measured with a circuit-test DMR-5200 handheld multimeter. Eight measurements were taken and a statistical analysis was performed to compare variance within each group and between groups.

The fibers were also tested in buffer using the same procedure. However, in order to do so, a basin of vacuum grease was placed around the body of the fiber leaving the two fiber ends protruding out and untouched by the grease. Then the basin was filled with PBS. Resistance measurements were taken one hour after filling the basin with PBS and 48 hours after. This was repeated three times with batches of three different fibers.

The results of the different fiber conductivities are presented in Table 1. The dual mechanical and conductive effect of having carbon nanotubes present in a material is essential for any composite. Electrical conductivity has been shown to support the growth of a variety of tissues such as cardiac muscle and neural tissue. Furthermore, it is key for neurite extension, where electrical propagation assists in the growth of neurons on carbon nanotube deposited planar substrates. The effect of which can be attributed to the carbon nanotubes acting as excellent free radical inhibitors. This is due in part to their ability to either donate or accept electrons. As such, free radicals which are considered detrimental to cell viability, are absent from the agarose fibers.

Dry samples of CNF prepared according to the present invention were shown to be electro-conductive with a specific conductivity of approximately 130-160 S cm⁻¹. These values fall near the range of specific conductivity of CNF prepared using the polymer PVA. In addition, the fibers were tested in buffer. The specific conductivity dramatically decreases in the pristine fiber when immersed in buffer by almost 2 orders of magnitudes, while the functionalized fibers show much less variation (LN+) and even no deterioration at all (CDAP+). This indicates that the cross-linking effect of the functionalization reaction impedes the swelling of the fiber that leads to a decrease in conductivity affecting electrical paths, which was seen in the pristine fibers.

TABLE 1
Specific conductivities of fibers in the dry state, and 1 hour and
48 hours after wetting. Conductivity retention in % is indicated
as well.

| | Specific Conductivity S cm⁻¹ | | | | |
|---|---|---|---|---|---|
| Fiber type | Dry | 1 h wet | Retention | 48 h wet | retention |
| Pristine | 191 ± 14 | 6 ± 1 | 3% | 3 ± 0 | 2% |
| LN+ | 145 ± 0 | 64 ± 4 | 44% | 67 ± 1 | 46% |
| CDAP+ | 131 ± 1 | 131 ± 4 | 100% | 135 ± 55 | 103% |

### Brain Tissue Biocompatibility

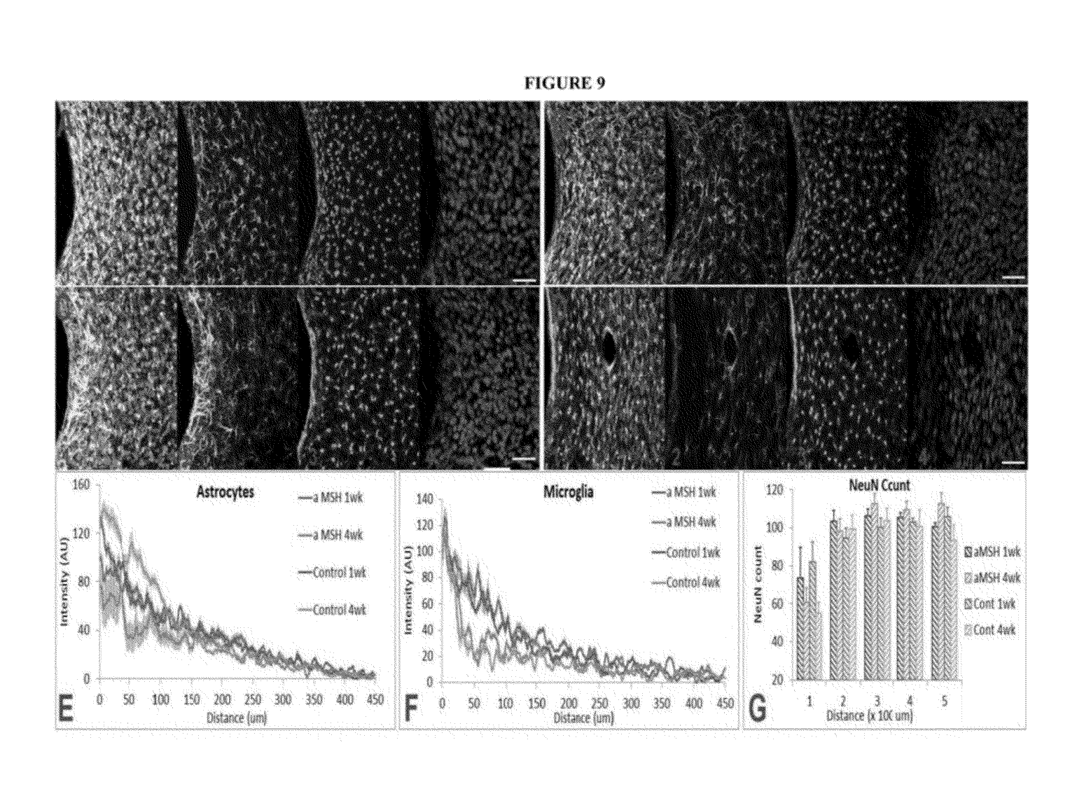

Initial evaluation to the effect of electrode biological functionalization on brain tissue in vivo was performed. Representative immunohistochemical images from 1 and 4 week implanted brain sites where pristine NCAC control (pristine) and alpha melanocyte stimulating hormone (α-MSH) activated fibers are shown in FIG. 9 along with their corresponding quantification of cellular response. FIG. 9(A) shows pristine fibers after one week of implantation; FIG. 9(B) shows α-MSH fibers after one week of implantation; FIG. 9(C) Pristine fibers after four weeks of implantation, and FIG. 9(D) α-MSH fibers after four weeks of implantation. In FIGS. 9(A)-(D): 1 designates merged cell responses, 2 designates astrocyte response, 3 designates microglia response, and 4 designates neuron response. Quantification of cell response as a function of distance from implant edge is shown in FIGS. 9(E)-(G) for astrocytes, microglia, and neurons respectively. A significant difference in the effect of the functionalization with α-MSII on the formation of the glial response (gliosis) and neural exclusion was observed. The use of other more specific adhesion molecules could prove to be more beneficial to neuronal survival and gliosis reduction.

### Mechanical Testing

Tensile properties of the CNT fibers were tested using an MTS model Sintech 5/D tension machine, fitted with the 100N load cell at room temperature with 50% relative humidity. A minimum of 5 fibers per sample were tested. To evaluate the effect of the activation on the agarose, samples were hydrated by immersing individual fibers in PBS at 50° C. (close to the agarose melting temperature) under gentle agitation for one hour. The mechanical testing was terminated when fibers reached their breakpoint.

The results of the mechanical tensile testing are shown in Table 1. Fiber stability was evaluated through hydration at a temperature close to the agarose melting point (50° C.). The dry fibers exhibited stiffness close to over 1 GPa, with the pristine fibers being the stiffest. All fibers exhibited a rigid and tough behavior, with none of them failing through a brittle manner, but rather maintaining their strength past the yield point till complete failure. Once hydrated, the CDAP functionalized fibers (LN+ and CDAP+) were evaluated and studied for their tensile properties. A 90% and 80% drop in the elastic modulus for the LN+ and CDAP+ respectively was observed for fibers in dry condition, accompanied with an decrease in yield and maximal strain. When CDAP is added to the agarose, cyano-ester termini results, and is available to react with free amide groups in the reaction. Competing reaction exists, where either a carbamate or an imidocarbonate can be formed from the cyanate ester. The latter forms either a cyclic bond within an agarose backbone or a crosslink between adjacent polymer chains, thus resulting in a slightly crosslinked and more stable CNT fiber (CDAP+). When laminin, a high molecular weight protein is added to the reaction (LN+), there is increased coupling, principally due to the available ε-amines of surface lysine, forming an isourea bond resulting in the observed CNF

stability. The late addition of the quenching ethanolamine to the functionalization reaction leads to elevated density of the crosslinking imidocarbonate in the CDAP+ fibers. Moreover, the crosslinking density of the CDAP+ fibers is higher than the LN+ samples because the distance between formed cross-linking junctions is shorter. The plasticization process occurring due to water absorption brings the fiber's strength and modulus much closer to that of inherent brain tissue, thus become more compliant compared to silicon neural devices. Applicants designed these fibers to be biological viable, conductive and supportive for soft tissue, but their use is not limited to only that application. Using a higher melting point agarose, with a higher molecular weight, could increase the strength of the composite fibers and vice versa. The chemical reaction itself through changes in reagent stoichiometry can be used to further modify the mechanical stability of the fibers in a biological environment.

TABLE 2
Tensile results for different agarose/SWNT fibers in dry and
hydrated states.

| | | | Yield | Yield | |
|---|---|---|---|---|---|
| | | Modulus | Stress | Strain | Max Strain |
| Sample | | (MPa) | (MPa) | (%) | (%) |
| Pristine | Dry | 1280 ± 386 | 17.3 ± 5.1 | 1.8 ± 0.8 | 8.3 + 2.0 |
| | Hydrated | 0 | 0 | 0 | 0 |
| LN+ | Dry | 867 ± 247 | 14.3 ± 4.8 | 1.9 ± 0.7 | 6.2 ± 2.5 |
| | Hydrated | 85.6 ± 12.8 | 0.1 ± 0 | 4.7 ± 2 | 4.8 ± 1.8 |
| CDAP+ | Dry | 1060 ± 698 | 5.2 ± 0.6 | 0.7 ± 0.5 | 8.9 ± 0.3 |
| | Hydrated | 220 ± 120 | 0.6 ± 0 | 4.2 ± 2.8 | 10.5 ± 4.2 |

### Cytotoxicity and Cell Attachment

Fibers were cut into 5 mm pieces with a razorblade and placed into the wells of a Costar 96-well tissue-culture treated polystyrene plate. The plate was sterilized for 1 h in UV. Four types of fibers were used: CDAP+, LN−, LN+, and pristine fibers. Rat astrocytes were cultured in DMEM (Invitrogen), 10% FBS (Atlanta Biologicals), 1% Penicillin/Streptomycin at 37° C., 5% CO₂. The cells were cultured to 90% confluence and then trypsinized, centrifuged, and the pellet re-suspended in media and the cells counted. 15,000 astrocytes were seeded into each well containing fiber and incubated for 18 hours at 37° C. 15,000 astrocytes were also added to the positive and negative control wells.

After 18 hours, the media was aspirated from each well and washed with PBS. A 1:10 dilution of Alamar Blue (ABD Serotec) to regular media was prepared and 100 ul of this mixture was added to each well. The cells were incubated for 5 hours at 37° C. and then a fluorescence measurement was recorded at 560 excitation and 590 emission using a Tecan Infinite M200 Fluorescent Plate Reader. The data obtained was normalized to the positive controls. To allow the evaluation of cell attachment on functionalized agarose CNT composites, dispersion films were prepared in the following manner: After sonication, 90 μL of CNT/agarose was sandwiched between two 12 mm glass cover slips. Once cooled, flat gel capsule were formed.

These capsules, with a composition similar to that of the fibers, underwent chemical modification in the same manner described for the fibers. Discs were placed in a 24 well plate, sterilized under UV for 15 minutes, then washed with serum free culture media. 100,000 primary rat astrocytes were seeded onto the disks and incubated for two hours to allow for cell attachment. Regular media was added to the wells containing the disks and the plates were incubated for three days. Afterward, the astrocyte-seeded disks were either (1) stained with Calcein AM (Invitrogen) followed by imaging using in the form of 3D data sets using a Leica SP2 confocal laser scanning inverted microscope with a 10× dry objective, or (2) fixed with 4% PFA for 15 minutes at 4 degrees Celsius. Following fixation, the cells were stained with 1:500 v/v Hoechst 33258 (Anaspec) and imaged using a Zeiss Axio Observer Fluorescent Microscope.

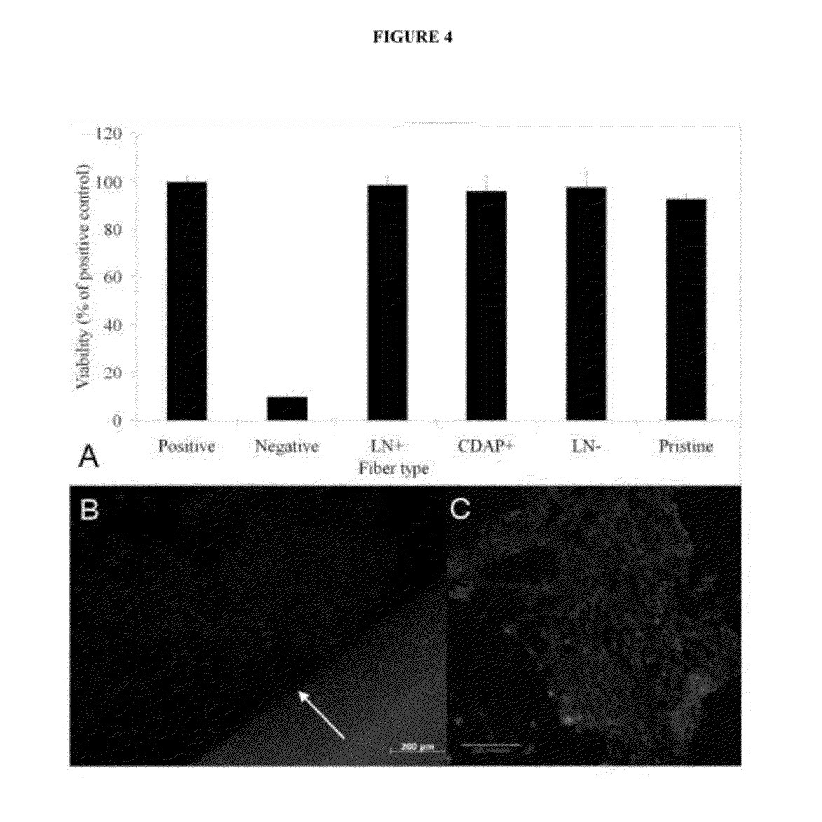

The metabolic activity of the cells exposed to different types of fibers was compared to positive-control cells kept in culture media. The effect of fiber presence on primary astrocyte culture viability is presented in FIG. 4a. Tests revealed that the fibers had no effect on the cell viability (p>0.05). An exception would be the pristine fibers, where a slight (10%) statistically different reduction in viability was observed (p<0.01). This reduction was due to presence of some catalyst residue in the CNT raw material. The process of functionalization, involving multiple washing steps, redeemed the processed fibers from these toxic residues.

Cell attachment studies performed on molded composite discs revealed that only the LN functionalized composites, seen in FIGS. 4b and 4c, allowed for cell attachment, while the control discs did not permit cell attachment. The agarose based materials maintain their biocompatibility properties, but are not permissive for cell attachment without the addition of cell adhesion moieties.

The process of conjugating peptides to the fabricated fibers was repeated several times successfully. It is a simple and safe process that does not require the use of a chemical hood or special safety measures. Moreover, the cytotoxicity and cell attachment studies performed on primary brain cells prove the process to be non-toxic to mammalian cells.

### In Vivo Characterization: Fiber Sterilization and Implantation

To allow accurate placement and smooth insertion of the fibers, a new insertion method developed by Applicants was used. First a 24 G×¾″ catheter (Terumo, Somerset, N.J.) was clipped. This allows the cannula and needle to be at the same length. The needle was withdrawn from the tip, and then the fiber was manually threaded into the now empty lumen tip. To insert the fibers into live tissue, the catheter was held above the insertion site using a mechanical arm, and a push of the needle drove the fiber into the required area without the needle penetrating the tissue. Prior to use, catheters with fibers were placed in self-sealing sterilizable pouches and sterilized with ethylene oxide gas (Anprolene; Anderson Products, Chapel Hill, N.C.) followed by 10 days aeration. Animal procedures were performed under the approval of the Wadsworth Center Institutional Animal Care and Use Committee (IACUC). Insertions were performed in a manner previously described (see D. H. Szarowski et al., Brain Res. 2003, 983, 23) with slight modifications. A 360 g male Sprague-Dawley rat was anesthetized with 2.5% isoflurane with oxygen (1 l/min) for 5 minutes in a pre-exposed chamber, and then maintained with 2% isoflurane with oxygen for the duration of the procedure (60 minutes) in a stereotaxic holder. Four holes were drilled using an electric drill (two on each side of midline, one anterior to bregma and one posterior to lambda). The dura was transected from the area of interest. Using a stereotactic holder, catheters were accurately placed above the insertion area, and a manual push of the needle allowed for smooth insertion of the fibers. Cellulose dialysis film (Fisher Scientific) was cut to 5×5 mm squares and applied over the exposed tissue and adhered to the skull. The skin was then closed using staples.

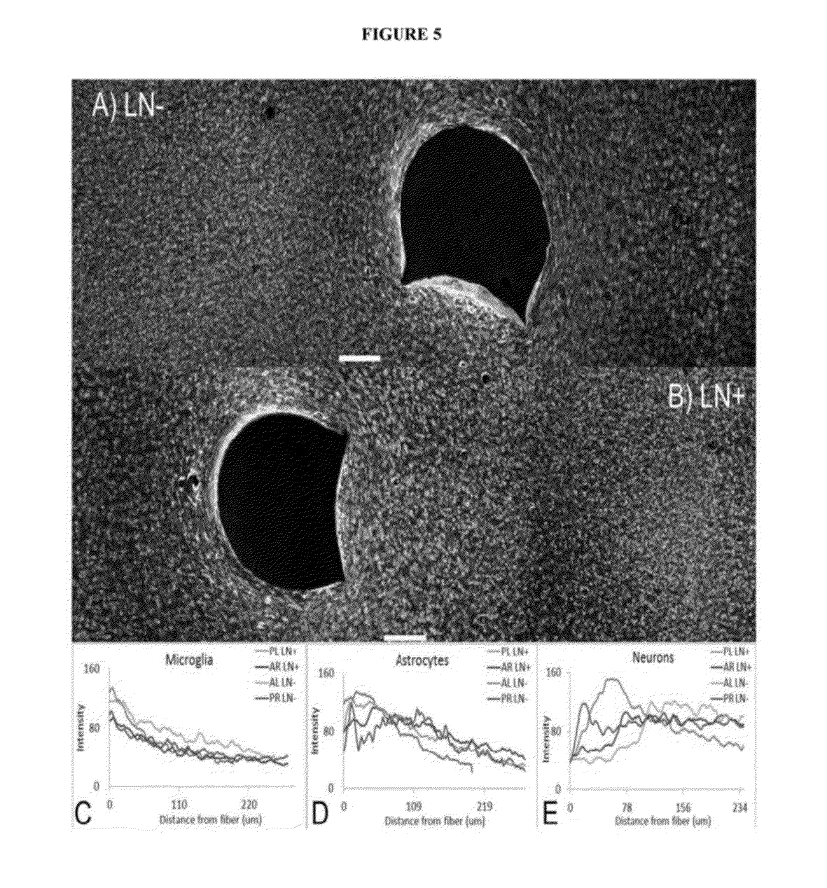

The insertion of fibers into a rat cerebral cortex was performed to allow preliminary evaluation of the insertion ability of the fibers into live tissue, and to acquire preliminary data with regard to the foreign body response inflicted by the presence of fibers in the tissue. Brain tissue inflammatory response to implanted materials is materialized through the presence of activated microglia and astrocytes at the vicinity of the implant site. Representative immunohistochemical images from sites where LN+ and LN− fibers were inserted into rat cortex are shown in FIGS. 5A and 5B. The intensities of astrocyte, microglia and neural expression measured for two of each fiber are shown in FIGS. 5C, 5D and 5E respectively.

The in vivo evaluation as to the effect of the inserted fibers on brain tissue does not reveal an effect of the functionalization with laminin on the formation of the glial response (gliosis). In both cases, a similar extent of activation of microglia and astrocytes is observed corresponding to the formation of mild gliosis. The resulting extent of glia activation (approximately 100 μm of glial sheath formation) is similar in extent to other biocompatible materials such as silicon. To reduce the extent of a glial response, LN can be tethered to silicone devices and implanted for four weeks. An extended period of implantation produces a reduction in the response as a result of the presence of the laminin functionalized nanofibers.

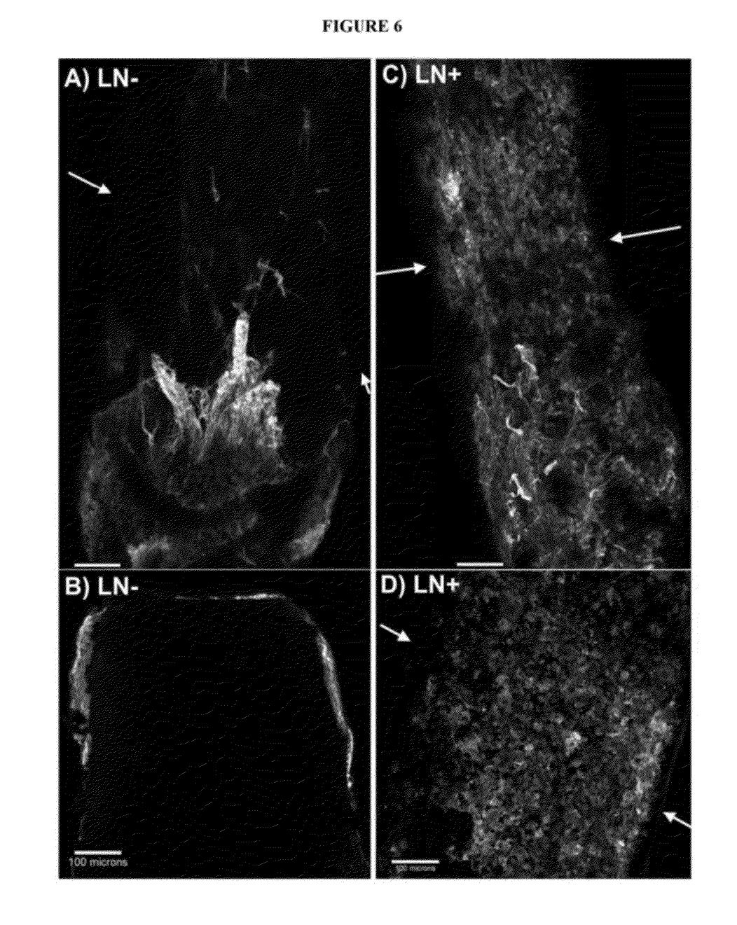

Representative images of fibers extracted from brain tissue are shown in FIG. 6. A difference between the fiber types could be observed once they were explanted. The laminin functionalized fibers promote more cell adhesion compared to the non-functionalized ones. Laminin is an ECM protein that is known to enhance neural growth both in vitro and in vivo. Naturally, the attachment enhancement properties of such constituent will have an effect on all cell types, as it is non-specific. Finer manipulation of the foreign body response to the fibers can be achieved by the addition of more specific adhesion molecules to the fibers. Examples include, but are not limited to, an inflammatory response reducing agent such as alpha melanocyte stimulating hormone or neuron specific adhesion molecules such as L1 molecule, shown to not only induce neurite outgrowth, but also reduce astrocytic attachment. Moreover, the explanted fibers demonstrated mechanical and dimensional stability. They became soft and pliable, in a trend similar to that shown with the mechanical tests.

### Tissue Processing and Immunohistochemistry

The animal was sacrificed by first anesthetizing with a ketamine/xylazine mixture, followed by transcardial perfusion. Tissue processing was performed based on standard immunohistochemistry (IHC) procedures. Horizontal 80-μm-thick tissue slices were cut using a vibratory microtome (Vibratom®, model 1000). Sections 900-1100 μm down from the dorsal surface of the brain were used. Once sectioning was completed, fibers remaining in the intact tissue were gently removed and processed similarly to the brain slices. Histochemistry was performed on tissue slices and fibers labeling 3 cell types. For primary antibodies the following reagents were used: (1) Astrocytes, rat anti-GFAP (Invitrogen, 13-0300, dilution 1:200) and (2) Microglia, rabbit anti-Ibal (019-19741, dilution 1:800, Wako, Richmond, Va. For secondary antibodies and added stain, the following reagents were used: (1) Goat anti-rabbit (Alexa Flour 488 A11008, dilution 1:200, Invitrogen), (2) Goat anti-rat (Alexa Flour 546 A 110081, dilution 1:200, Invitrogen), and (3) NeuroTrace stain for Nissl substance (530/615 N21482, Invitrogen). Sections were mounted on glass slides with ProLong Gold (Invitrogen) for confocal imaging. Histological images were collected in the form of 3D data sets using a Leica SP2 confocal laser scanning inverted microscope with a 10× dry objective. Images were stacked into X, Y projections of the entire Z dimension of the sample to allow for evaluation of cellular populations surrounding insertion sites. Images of the insertion site and two adjacent lateral fields were collected. Composite images were formed by aligning and superimposing through-focused projections of individual images using image-processing software (ImageJ, NIH). This allowed for observation of changes in immunohistochemistry immediately around the insertion sites and in control regions farther away. Fiber samples were imaged on both sides of the mounting slide because the black opaque nature of the fibers did not allow imaging of the full fiber thickness. One or two fields were collected for each side.

### Image Quantification

Using ImageJ, individual channels were converted to 8 bit, followed by correction of the background and intensity. A radial profile plugin was used to produce a profile plot of normalized integrated intensities around the implant site as a function of distance from the fiber center. The intensity gradient maximized at the fibers estimated edge is plotted for the implants.

Applicants have successfully fabricated agarose CNT hybrid fibers by taking advantage of agarose's ability to disperse and accommodate CNT's, its thermo-responsive hydrogelation and its functionalization potential. These fibers are rigid and tough when dry, but exhibit mechanical properties compliant with brain tissue once hydrated. They prove to be not just non-toxic, but biocompatible, and biologically modifiable. These properties, along with their stable electrical conductance, provide a novel material with use in neurophysiologic applications. While one aspect of the present invention was to produce fibers for implantable electrodes, the gelling properties of agarose allows it to be easily molded into other shapes with alternative applications such as directed nerve repair and nerve guidance conduit.

From the above description, it is understood that the present invention is well adapted to carry out the objects and to attain the advantages mentioned herein as well as those inherent in the invention. While presently preferred embodiments of the invention have been described for purposes of this disclosure, it will be understood that numerous changes may be made which will readily suggest themselves to those skilled in the art and which are accomplished within the spirit or the invention disclosed.

We claim:

1. A nanocomposite fiber comprising one or more carbon nanotubes encapsulated in an polysaccharide gel matrix.

2. The nanocomposite fiber of claim 1, wherein the polysaccharide is agarose.

3. The nanocomposite fiber of claim 1, wherein the carbon nanotube is a single wall carbon nanotube.

4. The nanocomposite fiber of claim 1, wherein the carbon nanotube is a multiwall carbon nanotube.

5. The nanocomposite fiber of claim 1, wherein the carbon nanotube-based fiber has a functionalized surface that allows for the covalent attachment of one or more bioactive substances.

6. The nanocomposite fiber of claim 4, wherein the bioactive substance is selected from the group consisting of proteins, peptides, glycogens and drugs.

7. The nanocomposite of claim 4, wherein the bioactive substance is selected from the group consisting of laminin, alpha melanocyte stimulating hormone, and L1 cell adhesion molecule.

8. The nanocomposite fiber of claim 1, wherein the fiber is loaded with at least one particle selected from the group consisting of platinum, palladium, gold, silver, titanium nitride, tantalum, tantalum oxide, iridium oxide and conductive polymers such as poly(3,4-ethylenedioxythiophene), polyimide, polyanyline, and polypyrole.

9. A method for fabricating a biocompatible carbon nanotube-based nanocomposite fiber, comprising:
a) preparing a liquid dispersion solution comprising carbon nanotubes and a polysaccharide;
b) injecting the liquid dispersion solution into a rotating bath of ethanol to form pre-fibers; and
c) drying the pre-fibers.

10. The method of claim 9, wherein the polysaccharide is agarose.

11. A method for fabricating a biocompatible carbon nanotube-based fiber, comprising:
a) preparing a liquid dispersion solution comprising carbon nanotubes and a polysaccharide;
b) injecting the liquid dispersion solution into a tube;
c) allowing the liquid dispersion to form a molded gel in the tube;
d) removing the molded gel from the tube.

12. The method of claim 1 wherein the polysaccharide is agarose.

13. A method for delivering a desired biomolecule to a subject comprising the steps of:
a) loading the biocompatible carbon nanotube-based fiber of claim 5 with a desired biomolecule; and
b) contacting said subject with the complexed carbon nanotube-based fiber.

14. The method of claim 13, wherein the polysaccharide is agarose.

15. The method of claim 14, wherein the loading of the polymeric nanoparticle carrier comprises covalently attaching the desired biomolecule to the agarose.

16. The method of claim 13, wherein the desired biomolecule is a drug.
